(19) United States
(12) Patent Application Publication
(10) Pub. No.: US 2008/0269533 A1
(43) Pub. Date: Oct. 30, 2008

(54) Method of selectively hydrogenating conjugated diene by using supported ionic liquid nano-pd catalyst

(75) Inventors: Jen-Chun Chang, Chiayi City (TW); Jann-Chen Lin, Chiayi City (TW); Chia-Ying Lee, Chiayi (TW); Guor-Tzo Wei, Chiayi (TW); Jung-Chung Wu, Chiayi City (TW)

Correspondence Address: BIRCH STEWART KOLASCH & BIRCH, PO BOX 747, FALLS CHURCH, VA, 22040-0747, US

(73) Assignee: Chinese Petroleum Corporation

(21) Appl. No.: 11/898,725

(22) Filed: Sep. 14, 2007

(30) Foreign Application Priority Data
Apr. 26, 2007 (TW) 096114822

(51) Int. Cl. C07C 5/03

(52) U.S. Cl. 585/277

(57) ABSTRACT

A method of selectively hydrogenating a conjugated diene by using a supported ionic liquid nano-palladium catalyst. The supported ionic liquid nano-palladium catalyst, hydrogen and a reactant having the conjugated diene react at a temperature ranging from 40 to 120° C. and a pressure ranging from 100 to 400 psig. A ratio of the catalyst to the reactant ranges from 1/20 to 1/250 (g/ml).

### CROSS REFERENCE TO RELATED APPLICATIONS

This Non-provisional application claims priority under 35 U.S.C. §119(a) on Patent Application No(s). 096114822 filed in Taiwan, Republic of China on Apr. 26, 2007, the entire contents of which are hereby incorporated by reference.

### BACKGROUND OF THE INVENTION

1. Field of Invention

The invention relates to a method of selectively hydrogenating a conjugated diene, and, in particular, to a method of selectively hydrogenating a conjugated diene by using a supported ionic liquid nano-palladium catalyst.

2. Related Art

An ethers compound, such as a methyl tert-butyl ether (MTBE) or a tertiary amyl methyl ether (TAME), is an octane booster that is greatly used in an unleaded gasoline, wherein TAME is made of a C5 fraction of a pyrolyzed gasoline as the feed, and the C5 fraction is formed in the naphtha pyrolysis process. The feed is first selectively hydrogenated so that the isoprene thereof is converted into the isopentene, which is etherealized with the methanol under the action of the acidic catalyst. The 2-methyl-2-butene (2M2B) and 2-methyl-1-butene (2M1B) pertain to the tertiary olefin, which may react with the methanol to form TAME, and the 3-methyl-1-butene (3M1B) does not have the activity.

In the feed specification of the TAME process, the diene value cannot exceed 1.5, and the content of the tertiary isopentene (isoamylene, 2-methyl-2-butene and 2-methyl-1-butene) has to fall within 19 to 30 wt %. Thus, how to process the C5 fraction of the pyrolyzed gasoline into the TAME feed meeting the specification is an important subject to be studied in the oil refining industry.

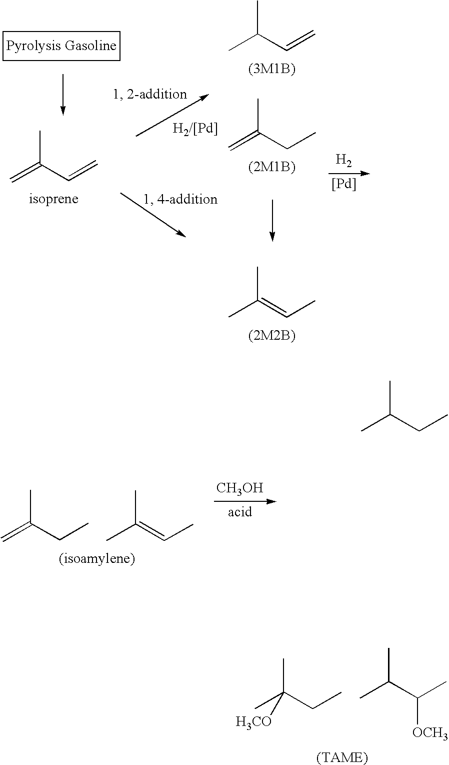

At present, two catalyst systems Ni/Al₂O₃ and Pd/Al₂O₃ exist in the current commercial process of selectively hydrogenating the pyrolyzed gasoline and have different operation conditions, as listed in Table 1.

TABLE 1
comparision between operation conditions in primary commercial
process of hydrogenating pyrolyzed gasoline.

Catalyst system

| | Ni/Al₂O₃ | Pd/Al₂O₃ |
|---|---|---|
| Reducing condition | | |
| Temperature | 430° C. | 100° C. |
| Pressure | 5.0 kg/cm² | 28.0 kg/cm² |
| Time | 14 hours | 18 hours |
| Reaction condition | | |
| Temperature | 80 to 140° C. | 60 to 120° C. |
| Pressure | 34.0 kg/cm² | 30 kg/cm² |
| Spatial flow speed | 1.23 | 3.5 |

In the Pd/Al₂O ₃ catalyst, the content of Pd is 0.3 wt % and the surface area of Pd/Al₂O₃ is 60 m² /g.

In addition, the typical commercial catalyst is manufactured by way of impregnation to obtain the metal oxide (e.g., NiO or PdO). The reducing process has to be performed before the reaction. When the pyrolyzed gasoline (containing about 10% of C5 olefin) serves as the feed, the conversion of the isoprene is about 90%, and the selectivity is about 90 to 92%. Thus, the conversion and the selectivity still have to be improved.

Therefore, it is an important subject of the invention to provide a method of selectively hydrogenating a conjugated diene for solving the above-mentioned problems.

### SUMMARY OF THE INVENTION

In view of the foregoing, the invention is to provide a method of selectively hydrogenating a conjugated diene by using a supported ionic liquid nano-palladium catalyst.

To achieve the above, the invention discloses a method of selectively hydrogenating a conjugated diene by using a supported ionic liquid nano-palladium catalyst. The supported ionic liquid nano-palladium catalyst, hydrogen and a reactant having the conjugated diene react at a temperature ranging from 40 to 120° C. and a pressure ranging from 100 to 400 psig. A ratio of the catalyst to the reactant ranges from 1/20 to 1/250 (g/ml).

As mentioned above, the invention can effectively perform the selectively hydrogenating reaction of the isoprene using the supported ionic liquid nano-palladium catalyst so as to form the 3-methyl-1-butene, 2-methyl-1-butene and 2-methyl-2-butene. Compared with the related art, the invention can achieve the catalysis of the activity and the selectivity using the combination of the ionic liquid, the nano-palladium metal and the carrier. One feature of the invention using the catalyst is to make the manufacturing of the nano-particles be independent from the coating step. Thus, the special nano-particles can be synthesized according to the requirements. The other feature of the invention is that the pre-reduction of the catalyst is not required, the product of the invention has the strong water resistance and is reusable, and the reaction activity and the selectivity are also better than those of the current commercial catalyst.

The invention will become more fully understood from the detailed description, which is given for illustration only, and thus is not limitative of the present invention.

### DETAILED DESCRIPTION OF THE INVENTION

The present invention will be apparent from the following detailed description, which proceeds with reference to the accompanying drawings, wherein the same references relate to the same elements.

The preferred embodiment of the invention provides a method of selectively hydrogenating a conjugated diene by using a supported ionic liquid nano-palladium catalyst. The supported ionic liquid nano-palladium catalyst, hydrogen and a reactant having the conjugated diene react at a temperature ranging from 40 to 120° C. and a pressure ranging from 100 to 400 psig. A ratio of the catalyst to the reactant ranges from 1/20 to 1/250 (g/ml).

The preferred reaction condition of selectively hydrogenating includes the temperature ranging from 60 to 80° C., the pressure ranging from 150 to 250 psig, and the ratio of the catalyst to the reactant ranging from 1/80 to 1/150 (g/ml).

In this embodiment, the reactant may be the piperylene, hexadiene, cyclopentadiene or cyclohexadiene.

Further explanation to the supported ionic liquid nano-palladium catalyst will be made in the following.

The nano-metal particle has the unique property especially in the aspect of the catalyzed reaction. However, because the nano-metal particles tend to flock together, a suitable stabilizer has to be added. The ionic type stabilizer may be, for example, the PVP (polyvinylpyrrolidone), the PVA (polyvinyl alcohol), the PAA (polyacrylic acid) or the PVE polyvinyl ether); the ligand type stabilizer may be, for example, the cyclodextrin or the phenanthroline; and the interfacial agent type stabilizer may be, for example, the CTAB (cetyltrimethylammonium bromide) and the TTAB (tetradecyltrimethylammonium bromide). Therefore, the nano-metal particles containing the stabilizer are suspended in the ionic liquid, and the ionic liquid is evenly covered on the carrier so that the supported ionic liquid nano-palladium catalyst may be manufactured.

The ionic liquid can stabilize the nano-particles and also enhance the selectivity of reaction according to the solubility difference between the diene type and the monoene type in the ionic liquid. The carrier functions to separate the ionic liquid effectively and to enlarge the contact surface with the reactant. Thus, the carrier has the biphasic function and the advantage of the fixed-bed reaction system and is relatively advantageous to the operation so that the selectively hydrogenating reaction of the isoprene can be effectively carried out.

The ionic liquid represents the substance composed of ions and exists in a liquid state at the low temperature (<100° C.). This feature is different from the typical molten salt. Replacing the conventional organic solvent with the ionic liquid to form the biphasic catalyzed reaction is advantageous to the recycling of the homogeneous phase catalyst. Meanwhile, the ionic liquid is advantageous to the separation of the product using the distillation method because it has no vapor pressure.

Consequently, the supported ionic liquid nano-palladium catalyst is manufactured by extracting the nano-palladium metal particles from the mixed solution of the palladous nitrate solution, the interfacial agent and the reductant using the ionic liquid in this embodiment, and the nano-palladium metal particle is supported on the carrier by way of impregnation.

The nano-palladium metal may be synthesized in the aqueous phase or the organic phase using the palladium-containing mineral salt or the organic metal complex in conjunction with the suitable amount of the stabilizer. Then, the produced nano-palladium particles are extracted out using the ionic liquid (e.g., [BMIM] [PF6]), which is immiscible to the water, and are then supported on the carrier such as aluminum oxide.

Herein, the content of the nano-palladium in the supported ionic liquid nano-palladium catalyst ranges from 0.02 to 1.0 wt % (0.05 to 0.5 wt % is preferred), the ratio of the ionic liquid to the carrier ranges from 5 to 400 microliters/g (10 to 200 microliter/g is preferred), the ratio of the nano-palladium to the stabilizer ranges from 1 to 1/40 (g/g) (1/2 to 1/15 is preferred), and the content of the nano-palladium in the ionic liquid ranges from 0.01 to 0.20 (g/ml), wherein the preferred content ranges from 0.02 to 0.10 (g/ml).

In addition, the reductant of this embodiment may be the boron hydrogen compound, alcohol, hydrazine, sodium citrate or ascorbic acid, and the stabilizer may be the polymeric type stabilizer, the ligand type stabilizer or the interfacial agent stabilizer.

Furthermore, the ionic liquid is composed of cations and anions, wherein the cation may pertain to the imidazole type [symbol] R₁R₂IM [symbol] ⁺, pyridine type [symbol] RP_{y} [symbol] ⁺, quaternary ammonium type [symbol] R₁R₂R₃R₄N [symbol] ⁺ or quaternary phosphorus type [symbol] R₁R₂R₃R4P [symbol] ⁺.

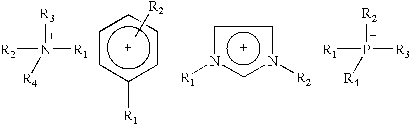

The anion may be BF₄⁻, PF₆⁻ or (CF₃SO₂)₂N⁻, which may form the water insoluble or non-organic phase insoluble ionic liquid with the cation. In addition, the carrier of this embodiment may be the Al₂O₃, SiO₂, SiO₂—Al₂O₃, TiO₂ or MCM-41 having the large surface area.

In order to make the feature of the invention become clearer, the experimental examples of the invention will be further described according to the non-limiting example.

First implemented model: manufacturing of supported ionic liquid nano-palladium catalyst.

In this model, a suitable amount of the palladous nitrate is dissolved in the deionized water, and a suitable amount of interfacial agent and a suitable amount of reductant are added and then stirred so that the nano-palladium metal particles can be obtained. Then, the nano-palladium metal particles are extracted out using the ionic liquid such as [BMIM] [PF6], and the particles are supported on the carrier such as the aluminum oxide by way of impregnation. Thus, the supported ionic liquid nano-palladium catalysts with different metal contents can be obtained.

Second experimental example: influence of reaction temperature.

An autoclave reactor is adopted to measure the activity of the reaction of selectively hydrogenating the isoprene. In this model, the reaction solution (120 ml) of the doped feed (20 vol % isoprene in n-heptane) is placed in the autoclave, and 1.5 g of the supported ionic liquid nano-palladium catalyst (0.3 wt % nano-Pd/IL/Al₂O₃) is added. Then, the hydrogen is introduced to react under the pressure of 200 psig for six hours. The reaction temperature ranges from 40 to 100° C., and the sample and analysis are taken every hours in the reaction procedure. The reaction results are listed in Table 2.

TABLE 2
influence of reaction temperature (six hours of reaction).

| Reaction | Conversion | Selectivity (%) | | | |
|---|---|---|---|---|---|
| temperature | (%) | Pentene | 3M1B | 2M1B | 2M2B |
| 40° C. | 23.4 | 100 | 23.2 | 24.6 | 52.2 |
| 60° C. | 99.0 | 99.9 | 16.4 | 26.1 | 57.4 |
| 80° C. | 100 | 99.8 | 1.8 | 17.6 | 80.5 |
| 100° C. | 100 | 87.6 | 0.4 | 12.0 | 75.3 |

According to the results, the conversion of 100% may be reached over 60° C., the pentene selectivity of 100% may be maintained at 80° C., and the selectivity is decreased to 87.6% at the reaction temperature of 100° C. after six hours of reaction. Meanwhile, when the reaction temperature is increased from 60° C. to 80° C., 3M1B and 2M1B in the product are significantly decreased and are then converted into 2M2B, wherein 3M1B cannot serve as the feed for the TAME synthesis. So, the production of 3M1B has to be possibly decreased. Consequently, it is obtained that the reaction temperature preferably ranges from 60 to 80° C.

Third experimental example: influence of content of nano-palladium metal.

The content of Pd in the nano-Pd/IL/Al₂O₃ catalyst is changed between 0.05 wt %, 0.1 wt %, 0.2 wt % and 0.3 wt %, and the activity and the selectivity thereof are respectively measured under the reaction condition of 200 psig, 60° C., 2.0 g catalyst/120 ml reaction solution (20 vol % isoprene in n-heptane). The results after six hours of reaction are listed in Table 3.

TABLE 3
influence of content of nano-palladium metal.

| | | Content of Pd (wt %) | | | | | |
|---|---|---|---|---|---|---|---|
| | | 0.05 | | 0.1 | | 0.3 | |
| | | 2 hr | 6 hr | 2 hr | 6 hr | 2 hr | 6 hr |
| Conversion (%) | | 20.4 | 96.7 | 80.9 | 100 | 55.9 | 100 |
| Selectivity (%) | pentene | 100 | 100 | 99.9 | 99.8 | 99.9 | 99.8 |
| | 3M1B | 24.9 | 22.4 | 19.7 | 8.1 | 19.7 | 2.5 |
| | 2M1B | 26.3 | 26.9 | 26.9 | 24.9 | 26.0 | 19.8 |
| | 2M2B | 48.9 | 50.7 | 53.3 | 66.9 | 54.2 | 77.5 |

According to the results, the 0.1 wt % catalyst oppositely has the higher activity than the 0.3 wt % catalyst, but 3M1B and 2M1B cannot be easily isomerized into 2M2B. In addition, the activity of 0.05 wt % Pd catalyst is insufficient. Although the selectivity of pentene is still 100%, the production of 3M1B reaches 22.4%. So, the ideal catalyst is 0.1 to 0.2 wt % of Pd/IL/Al₂O₃.

Fourth experimental example: comparison between supported nano-palladium and supported non-nano-palladium.

The supported non-nano-palladium catalyst with 0.3 wt % is manufactured by way of the typical impregnation to obtain Pd/Al₂O₃ after drying and calcination are performed, wherein the content of Pd is 0.3 wt %. Before the reaction, H₂ is first introduced under the conditions of the temperature of 140° C. and the pressure of 200 psig, and the reducing process is performed for four hours. Then, the reaction solution is added, and the activity test is performed under the reaction conditions the same as those of the third experimental example. The method of manufacturing the supported nano-palladium catalyst, 0.3 wt % of nano-Pd/Al₂O₃, is similar to that of the first experimental example, and the reaction results are listed in Table 4.

TABLE 4
comparison between supported nano-palladium and supported
non-nano-palladium.

| Catalyst | |
|---|---|
| Supported | Supported |
| nano- | non-nano- |
| palladium | palladium |

| | 2 hr | 6 hr | 2 hr | 6 hr |
|---|---|---|---|---|
| Conversion (%) | 60.9 | 97.8 | 90.7 | 100 |

| | | | | | |
|---|---|---|---|---|---|
| Selectivity (%) | pentene | 100 | 99.7 | 98.4 | 50.4 |
| | 3M1B | 23.4 | 5.6 | 3.3 | 0 |
| | 2M1B | 27.3 | 22.5 | 13.0 | 0 |
| | 2M2B | 49.3 | 71.6 | 82.0 | 50.4 |

According to the results, the non-nano-palladium catalyst has the relatively high hydrogenating activity but the poor isopentene selectivity (only 50% of selectivity is left after six hours), and the nano-palladium catalyst still has the pentene selectivity of 99.7% under the conversion of 97.8%. However, the production of the 3M1B, which cannot be synthesized into TAME, is still high.

Fifth experimental example: comparison between supported ionic liquid nano-palladium and supported non-ionic liquid nano-palladium.

The method of manufacturing the supported non-ionic liquid nano-palladium (nano-Pd/Al₂O₃) is similar to that of the fourth experimental example, and the cycle test method is adopted to measure the activity and the stability of the catalyst. In this method, six hours of reaction is performed at 60° C., then the new reaction solution reacts for six hours at 80° C., and finally the new reaction solution again reacts at 60° C. for six hours. The results are listed in Table 5.

TABLE 5
comparison between supported ionic liquid nano-palladium and
supported non-ionic liquid nano-palladium.

| | | Catalyst | | | | | |
|---|---|---|---|---|---|---|---|
| | | Supported non-ionic liquid | | | Supported ionic liquid | | |
| | | nano-palladium | | | nano-palladium | | |
| | | | | 60° C. | | | 60° C. |
| | | 60° C. | 80° C. | (Second time) | 60° C. | 80° C. | (Second time) |
| Conversion (%) | | 97.8 | 100 | 85.3 | 100 | 99.4 | 100 |
| Selectivity (%) | pentene | 99.7 | 99.6 | 99.9 | 99.8 | 99.8 | 99.8 |
| | 3M1B | 5.6 | 0 | 15.4 | 2.5 | 1.20 | 3.7 |
| | 2M1B | 22.5 | 19.0 | 26.5 | 19.8 | 16.5 | 20.2 |
| | 2M2B | 71.6 | 85.6 | 58.0 | 77.5 | 82.1 | 75.9 |

According to the results, the supported ionic liquid nano-palladium catalyst and the supported non-ionic liquid nano-palladium catalyst have the approximate activities, and the better pentene selectivities, but more 3M1B is produced using the non-ionic liquid nano-palladium catalyst, and the production of the isoamylene (2M1B and 2M2B) is lower. According to the cycle test results, after the non-ionic liquid nano-palladium reacts at 80° C., the hydrogenating activity is reduced, and the 3M1B isomerization is also reduced. This might be caused by the flocking of the nano-palladium metal, and the ionic liquid nano-palladium is free from this phenomenon.

Six experimental example: comparison between supported and non-supported ionic liquid nano-palladium catalysts.

0.3 wt % nano-Pd/IL and 0.3 wt % nano-Pd/IL/Al₂O₃ catalyst react under the reaction conditions the same as those of the third experimental example, and the activity test is performed. The results after six hours of reaction are listed in Table 6.

According to the result, the activity and the selectivity of the supported ionic liquid nano-palladium catalyst are far higher than those of the non-supported ionic liquid nano-palladium (Biphasic system). That is, immersing and distributing the ionic liquid over the Al₂O₃ carrier are advantageous to the contact with the reactant.

TABLE 6
comparison between supported and non-supported ionic liquid
nano-palladium.

| | | Catalyst | |
|---|---|---|---|
| | | Supported ionic liquid | Non-supported ionic liquid |
| | | nano-palladium | nano-palladium |
| Conversion (%) | | 99.6 | 76.4 |
| Selectivity | pentene | 99.8 | 99.9 |
| (%) | 3M1B | 2.7 | 23.8 |
| | 2M1B | 21.1 | 25.9 |
| | 2M2B | 76.0 | 50.3 |

Seventh experimental example: influence of usage of ionic liquid.

After the nano-palladium particle is manufactured using the aqueous phase, the ionic liquid is extracted out so that the solvent is volatilized and the ionic liquid with the solvent being volatilized is coated over the carrier such as Al₂O₃, wherein the usage of the ionic liquid influences the action of the interfacial agent and the thickness of the ionic liquid on the Al₂O₃. The activity test is performed under the reaction conditions the same as those of the third experimental example, and the results are listed in Table 7. According to the results, it is obtained that when the usage of the ionic liquid is halved relative to the fifth experimental example, the reaction activity is oppositely increased and the production of 3M1B becomes less, that is, the production of the isoamylene is high. According to the cycle test result, the phenomenon of the flocked nano-palladium particles does not occur.

TABLE 7
influence of usage of ionic liquid.

| | | | Catalyst | | | |
|---|---|---|---|---|---|---|
| | | | 0.3 wt % | | 0.3 wt % | |
| | | | nano-Pd/ | | nano-Pd/ | |
| | | | IL/Al₂O₃ | | 0.5IL/Al₂O₃ | |
| | | | Reaction time | | | |
| | | | 2 hr | 6 hr | 2 hr | 6 hr |
| 60° C. | Conversion (%) | | 55.9 | 100 | 64.9 | 99.2 |
| | Selectivity (%) | pentene | 99.9 | 99.8 | 99.9 | 99.6 |
| | | 3M1B | 19.7 | 2.5 | 18.0 | 0.5 |
| | | 2M1B | 26.0 | 19.8 | 25.0 | 10.6 |
| | | 2M2B | 54.2 | 77.5 | 56.9 | 88.6 |
| 80° C. | Conversion (%) | | 60.5 | 99.4 | 73.6 | 99.1 |
| | Selectivity (%) | pentene | 99.9 | 99.8 | 99.9 | 99.7 |
| | | 3M1B | 17.2 | 1.2 | 15.2 | 0.7 |
| | | 2M1B | 24.7 | 16.5 | 23.1 | 11.0 |
| | | 2M2B | 58.0 | 82.1 | 11.6 | 89.9 |
| 60° C. | Conversion (%) | | 56.9 | 100 | 65.4 | 100 |
| | Selectivity | pentene | 100 | 99.8 | 99.9 | 99.7 |
| | | 3M1B | 22.2 | 3.7 | 18.0 | 0.8 |
| | | 2M1B | 26.0 | 20.2 | 25.3 | 13.9 |
| | | 2M2B | 51.8 | 75.9 | 56.6 | 85.1 |

Eighth experimental example: influence of usage of nano-palladium particle stabilizer.

Different amounts of stabilizers (TTAB) are used to manufacture the 0.3 wt % nano-Pd/IL/Al₂O₃ catalysts, the cycle test method of the fifth experimental example is adopted to measure the activity and the stability of the catalyst, and the results are listed in Table 8.

TABLE 8
influence of usage of stabilizer.

| | | Catalyst | | | | | |
|---|---|---|---|---|---|---|---|
| | | 6.7 g TTAB/gPd | | | 13.7 g TTAB/gPd | | |
| | | | | 60° C. | | | 60° C. |
| | | 60° C. | 80° C. | (Second time) | 60° C. | 80° C. | (Second time) |
| Conversion (%) | | 100 | 100 | 92.9 | 100 | 95.8 | 58.4 |
| Selectivity (%) | pentene | 99.6 | 99.6 | 99.7 | 99.9 | 99.9 | 100 |
| | 3M1B | 0 | 0 | 3.8 | 14.0 | 16.8 | 20.6 |
| | 2M1B | 14.4 | 12.5 | 24.2 | 26.5 | 24.9 | 21.4 |
| | 2M2B | 85.2 | 87.1 | 71.7 | 59.4 | 58.3 | 58.0 |

According to the results, it is obtained that the activity obtained using the catalyst of 13.4 g TTAB/gPd is worse than that obtained using 6.7 g TTAB/gPd, and the production ratio difference of 3M1B is greater. Meanwhile, it is also found, according to the cycle test result, that the phenomenon of the significant activity deterioration occurs when a larger amount of TTAB stabilizer is used.

In this invention, the supported ionic liquid nano-palladium catalyst is manufactured according to the properties of the nano-palladium metal, the ionic liquid and the carrier. According to the results of the examples, this series of catalyst can be effectively applied to the selectively hydrogenating reaction of the isoprene in the C5 fraction of the pyrolyzed gasoline in order to form the feed for the synthesis of TAME. The feed is the tertiary isopentene (2M2B and 2M1B) and has the reaction activity and the selectivity which may reach about 100% that is far higher than 90% in the commercial manufacturing process.

In summary, the invention can effectively perform the selectively hydrogenating reaction of the isoprene using the supported ionic liquid nano-palladium catalyst so as to form the 3-methyl-1-butene, 2-methyl-1-butene and 2-methyl-2-butene. Compared with the related art, the invention can achieve the catalysis of the activity and the selectivity using the combination of the ionic liquid, the nano-palladium metal and the carrier. One feature of the invention using the catalyst is to make the manufacturing of the nano-particles be independent from the coating step. Thus, the special nano-particles can be synthesized according to the requirements. The other feature of the invention is that the pre-reduction of the catalyst is not required, the product of the invention has the strong water resistance and is reusable, and the reaction activity and the selectivity are also better than those of the current commercial catalyst.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A method of selectively hydrogenating a conjugated diene by using a supported ionic liquid nano-palladium catalyst, wherein the supported ionic liquid nano-palladium catalyst, hydrogen and a reactant having the conjugated diene react at a temperature ranging from 40 to 120° C. and a pressure ranging from 100 to 400 psig, and a ratio of the catalyst to the reactant ranges from 1/20 to 1/250 (g/ml).

2. The method according to claim 1, wherein the supported ionic liquid nano-palladium catalyst is prepared by using an ionic liquid to extract nano-palladium metal particles from a mixed solution of an interfacial agent, a reductant and a solution of palladous nitrate, and by supporting the nano-palladium metal particles on a carrier by way of impregnation.

3. The method according to claim 2, wherein a content of nano-palladium in the supported ionic liquid nano-palladium catalyst ranges from 0.02 to 1.0 wt %, a ratio of the ionic liquid to the carrier ranges from 5 to 400 (microliters/g), a ratio of the nano-palladium to a stabilizer ranges from 1 to 1/40 (g/g), and a content of the nano-palladium in the ionic liquid ranges from 0.01 to 0.20 (g/ml).

4. The method according to claim 1, wherein a reaction condition of selectively hydrogenating comprises a temperature ranging from 60 to 80° C., a pressure ranging from 150 to 250 psig, and a ratio of the catalyst to the reactant ranging from 1/80 to 1/150 (g/ml).

5. The method according to claim 2, wherein a content of nano-palladium in the ionic liquid ranges from 0.02 to 0.10 (g/ml).

6. The method according to claim 2, wherein the nano-palladium metal particles are synthesized in an aqueous phase or an organic phase using a palladium-containing mineral salt or an organic metal complex in conjunction with a stabilizer, nano-palladium metal particles are formed and extracted out of the ionic liquid, and the nano-palladium metal particles are supported on the carrier.

7. The method according to claim 2, wherein the reductant is a boron hydrogen compound, an alcohol, hydrazine, sodium citrate or ascorbic acid.

8. The method according to claim 3, wherein the stabilizer is a polymeric type stabilizer, a ligand type stabilizer or an interfacial agent type stabilizer.

9. The method according to claim 2, wherein the ionic liquid is composed of cations and anions.

10. The method according to claim 9, wherein the cation pertains to an imidazole type, a pyridine type, a quaternary ammonium type or a quaternary phosphorus type.

11. The method according to claim 9, wherein the anion is BF₄⁻, PF₆⁻or (CF₃SO₂)₂N⁻.

12. The method according to claim 2, wherein the carrier is Al₂O₃, SiO₂, SiO₂—Al₂O₃, TiO₂ or MCM-41.

13. The method according to claim 1, wherein the reactant is piperylene, hexadiene, cyclopentadiene or cyclohexadiene.

14. The method according to claim 1, wherein a content of palladium metal in the catalyst ranges from 0.05 to 0.5 wt %.

15. The method according to claim 2, wherein a ratio of the ionic liquid to the carrier ranges from 10 to 200 (microliters/g).
